(19) United States
(12) Patent Application Publication
(10) Pub. No.: US 2008/0127400 A1
(43) Pub. Date: Jun. 5, 2008

(54) Protective Mask

(75) Inventor: Yves Dupuis, St-Thomas de Joliette (CA)

Correspondence Address: Miltons LLP, 225 Metcalfe Street, Suite 700, Ottawa, ON, K2P 1P9, omitted

(73) Assignee: SNC TECHNOLOGIES INC., Le Gardeur, QC (CA)

(21) Appl. No.: 11/570,286

(22) PCT Filed: Jun. 10, 2005
(86) PCT No.: PCT/CA05/00898

§ 371 Date: Nov. 1, 2007

(30) Foreign Application Priority Data
Jun. 11, 2004 (CA) 2470755

(51) Int. Cl. A42B 1/08; A61F 9/02; A41D 13/00; A61C 5/14

(52) U.S. Cl. 2/424; 2/429; 2/445; 2/9; 128/859

(57) ABSTRACT

A protective facemask for use in simulated combat with nonlethal ammunition is modular in form. Its primary elements including a mouthpiece protecting portion with cheek guards, a lower bridge connected along the upper edge of the mouthpiece to serve as the deflector against projectiles penetrating between the mouthpiece and eye goggles, and a forehead bridge to serve as a deflector against projectiles penetrating between the eye goggles and a helmet to be worn by the user. These components are held in place by being fastened to around the chin and neck of a wearer.

### FIELD OF THE INVENTION

This invention relates to a protective mask to be worn by persons playing a simulated game of combat with firearms. More particularly, it applies to a mask suitable for use when players are utilizing low energy, nonlethal ammunition

### BACKGROUND TO THE INVENTION

The recreational game of “Paint Ball” has become established generally in North America. In this game participants use low energy, pseudo-firearms to compete in a simulated combat environment. In parallel, military, police, and other government agencies have come to appreciate the importance of allowing personnel to utilize their standard weapons in combat simulating training.

A class of low energy ammunition has been developed which operates in standard weapons. This low energy ammunition, exemplified by U.S. Pat. No. 5,359,937, the contents of which are adopted herein by reference, is capable of firing a round which essentially contains a marking liquid in a soft polymer shell. Such low energy rounds are virtually incapable of causing a fatality. However, they can cause injury if they strike a vital portion of a person's body, such as an eye, mouth, ear or throat. Accordingly, it is appropriate to wear a protective mask during such activities.

This application addresses such a form of protective mask.

The invention in its general form will first be described, and then its implementation in terms of specific embodiments will be detailed with reference to the drawings following hereafter. These embodiments are intended to demonstrate the principle of the invention, and the manner of its implementation. The invention in its broadest and more specific forms will then be further described, and defined, in each of the individual claims which conclude this Specification.

### SUMMARY OF THE INVENTION

According to one variant of the invention, a protective mask comprises:

1) a mouthpiece portion for covering the nose, mouth and cheeks of a wearer;

2) eye goggles for covering the eyes of a wearer; and

3) a first bridging portion interconnecting the lower edge of the eye goggles with the upper edge of the mouthpiece portion and serving as a deflector against the entry of projectiles between the eye goggles and the mouthpiece portion,

the mouthpiece portion, eye goggles, and first bridging portion all being provided with fastening means by which such components may be reversibly assembled together as required by a user.

While reference is made to “eye goggles”, this expression includes eye goggles fitted with a frame to enhance the connection of components.

Optionally, but preferably, a second bridging portion dimensioned to overlie the forehead of a wearer is provided together with interconnecting fastening means by which the second bridging portion may be reversibly connected to along the upper portion of the eye goggles. This second bridging portion may also interfere it with the visor or rim of a helmet to be worn by the user.

Preferably, the mouthpiece portion is perforated to allow for the passage of air. These perforations may be formed in detachable plates which may be connected by fasteners to their position on the mouthpiece.

The above components are suited for use with a chin and neck-embracing collar, preferably of a textile material which is shaped to engage and be fastened around the neck and back of the head of a wearer. Optionally but preferably, over-the-head-strap means interconnect ear-covering portions of the collar, passing over the crown of the head of a wearer. Such straps may form part of a pad in crossing over the top of the head. The mouthpiece portion may connect to this ear-covering portion of the collar.

The foregoing summarizes the principal features of the invention and some of its optional aspects. The invention may be further understood by the description of the preferred embodiments, in conjunction with the drawings, which now follow.

### BRIEF DESCRIPTION OF THE DRAWINGS

FIG. 1 is an isometric view of the head of a person wearing the mask system according to a preferred variant of the invention.

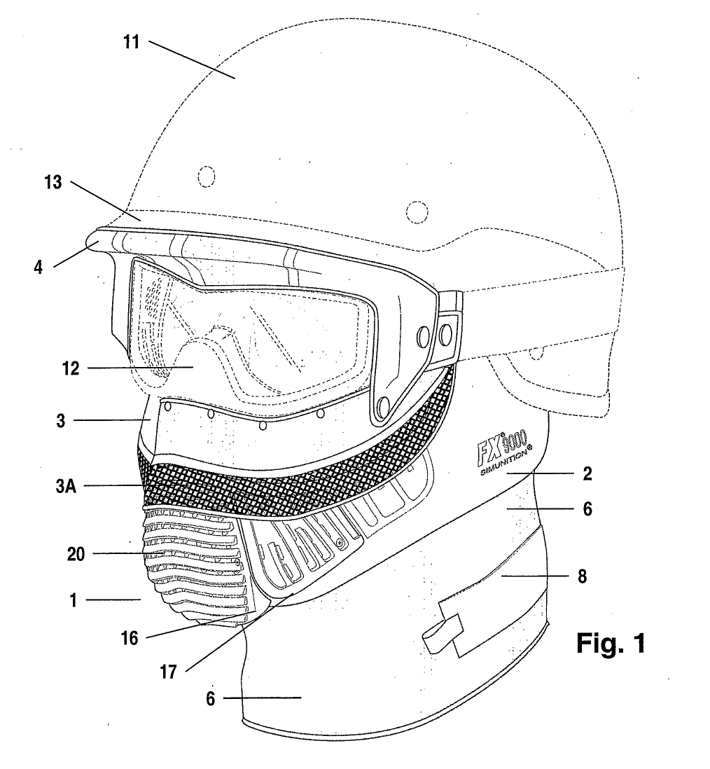

FIG. 2 is a front view of the mask of FIG. 1.

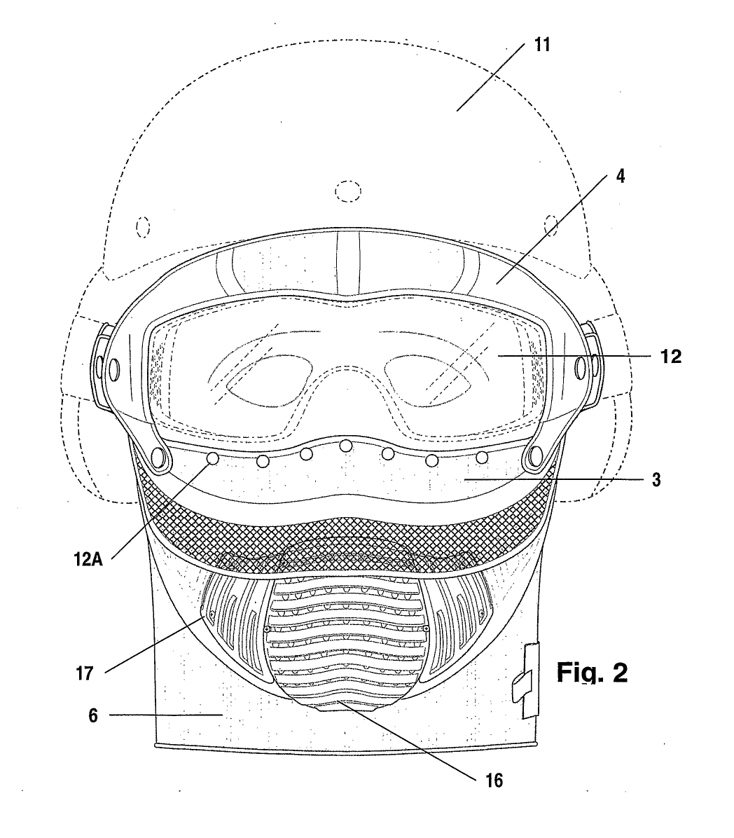

FIG. 3 is a right side view of the mask of FIG. 1.

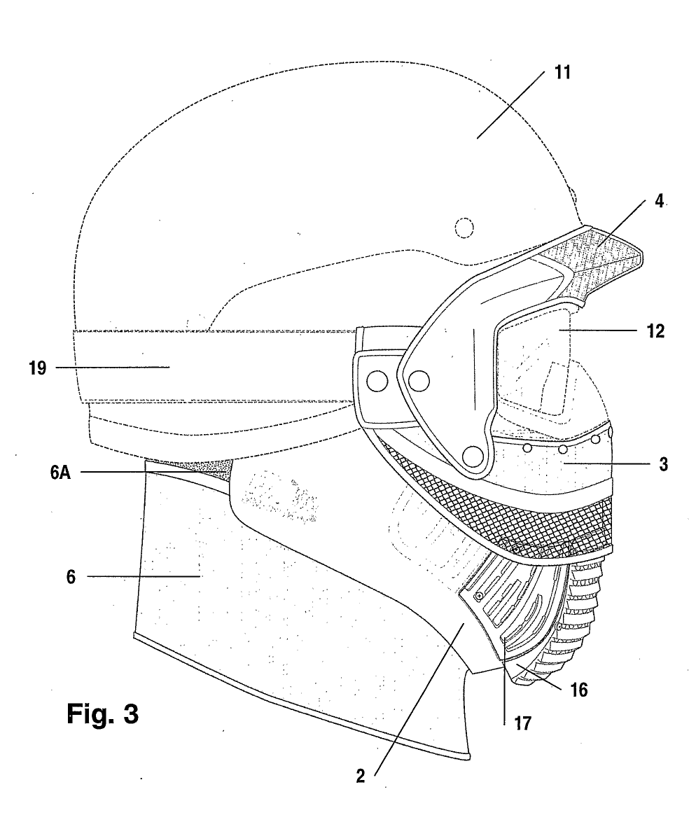

FIG. 4 is an exploded view of the components of the preferred variant of the invention of FIGS. 1-3.

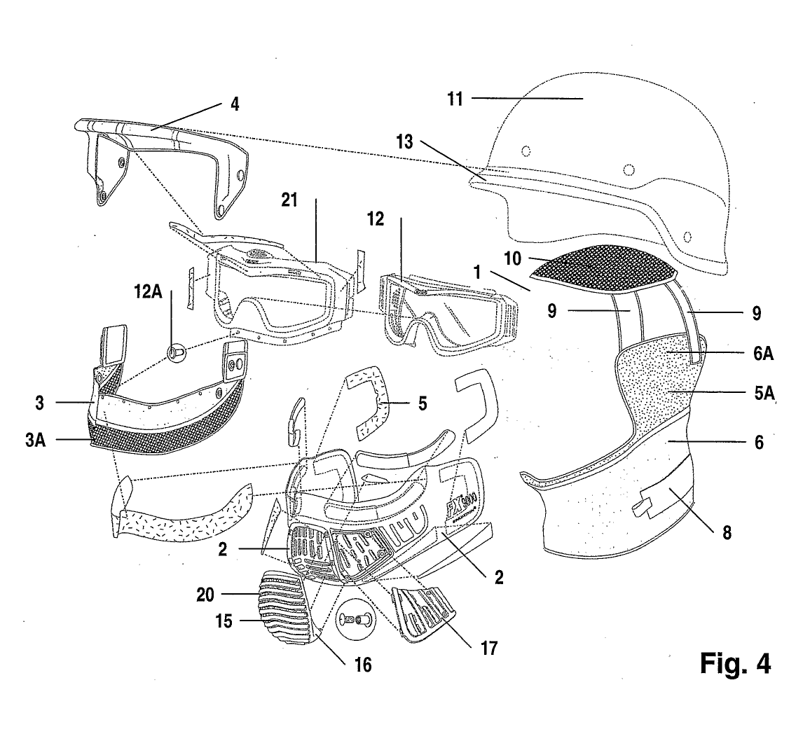

### DESCRIPTION OF THE PREFERRED EMBODIMENT

As seen in the Figures, a mask system is provided for the head 1 of a wearer which has a mouthpiece portion 2, a protective nose bridge 3 and a protective forehead bridge 4. The nose bridge 3 may have a flexible fabric portion 3A for ventilation.

The mouthpiece portion 2 is held onto the face of a wearer by the attachment of the mouthpiece portion 2 through fastening means 5, such as hook-and-loop fasteners, (similar to that sold under the trademark Velcro), to attachment points 5A on an ear-covering extension 6A of the textile-based collar 6. This collar 6 preferably has a portion 7 that passes beneath the chin, covering the throat of a wearer. The collar 6 is also includes to a neck-wrapping portion that may be fastened securely around the neck of a wearer by fastener 8.

The collar 6 may also be provided with straps 9 extending over the head of a wearer, preferably connecting to a top-of-the-head fabric pad 10 that provides a seat for a helmet 11 to be worn by a wearer. The straps 9 may commence from portions 6A of the collar 6 that overlie the ears of a wearer.

The mouthpiece portion 2 is shaped to fit at a spaced but slight separation from the lower portion of eye goggles 12. The eye goggles 12 are held in place by a strap 19. An additional frame 21 which fits intimately over the goggles 12 carries fastening means and functions as part of the goggles 12. The protective nose bridge 3 spans the space between the goggle frame 21 covering the goggles 12, and the mouthpiece portion 2. This serves as a lower deflector to prevent entry of projectiles between the mouthpiece portion 2 and goggles 12.

Generally, all components are interengaged by fasteners. The fasteners may be of various types, including hook-and-loop fasteners, threaded fasteners, pins and snap fasteners. Specific fastening means 12A is employed along the lower edge of the goggle frame 21 enclosing goggles 12 and the upper edge of the protective nose bridge 3.

Additionally, a similar fastening means is preferably present along the inner edge of the protective forehead bridge 4 and upper edge of the goggle frame 21. This forehead bridge 4 is intended to fit against the visor portion 13 of the helmet 11 overlying or inter-fitting with the visor 13. This further bridge 4 serves to deflect higher-level entry of projectiles between the goggles 12 and helmet 11.

The mouthpiece portion 2 is provided with apertures 15 to allow air to enter for the purposes of breathing. For convenience of construction, these apertures 15 may be provided by plates 16, 17 attached respectively over the mouth, left and right cheek portions of the mouthpiece 2. Optionally, a filter means, such as a gauze or screen (not shown), may be fitted against the inside surfaces of these plates 16, 17 to exclude dust, insects and the like.

The mask system of the invention is intended, for convenience, to adapt to typical goggles 12 of the type generally available for skiers. Such goggles 12 are normally ventilated to minimize the formation of condensation on the inner surface of the lens portions of the goggles. By incorporating such goggles into a mask system in accordance with the invention having separate ventilation apertures 20, the prospects that exhaled air from the wearer will enter the inner side of the goggles 12 and fog the lens portions are reduced.

Further, by reason of its modular character, portions of the mask system may be readily removed during exercises when training activities have been suspended. Thus the bridges 3, 4, goggles 12 and mouthpiece 2, together with the helmet 11, may be progressively removed with the wearer retaining the collar 6 and its associated components. This reduces the discomfort of having to wear the mouthpiece 2 and goggles 12 continuously. At the same time, these removed components may be readily refitted in place for resumption of combat simulation activities.

### CONCLUSION

The foregoing has constituted a description of specific embodiments showing how the invention may be applied and put into use. These embodiments are only exemplary. The invention in its broadest, and more specific aspects, is further described and defined in the claims which now follow.

These claims, and the language used therein, are to be understood in terms of the variants of the invention which have been described. They are not to be restricted to such variants, but are to be read as covering the full scope of the invention as is implicit within the invention and the disclosure that has been provided herein.

The embodiments of the invention in which an exclusive property or privileges claimed are defined as follows:

1. A protective mask comprising:
1) a mouthpiece portion for covering the nose, mouth and cheeks of a wearer;
2) eye goggles for covering the eyes of a wearer;
3) a first bridging portion for interconnecting between the lower edge of the eye goggles and the upper edge of the mouthpiece portion to serve as a deflector against the entry of projectiles between the eye goggles and the mouthpiece portion,
the mouthpiece portion, eye goggles, and first bridging portion all being provided with first fastening means by which such a components may be reversibly assembled together as required by a user.

2. A protective mask as a claim 1 comprising:
1) a second bridging portion dimensioned to overlie the forehead of a wearer;
2) second fastening means by which the second bridging portion may be reversibly connected along the upper edge portion of the eye goggles.

3. A protective mask as in claim 1 wherein said mouthpiece portion is provided with detachable plates which are perforated to allow for the passage of air therethrough.

4. A protective mask as in any one of claims 1, 2 or 3 in combination with a chin and neck embracing collar which is shaped to engage and be fastened around the neck of a wearer, said collar being provided with fastener means for connecting it to said mouthpiece.

5. A protective mask as in claim 4 wherein said collar includes ear-covering portions, said mask further comprising over-the-head-strap means interconnected with the ear-covering portions of the collar by passing over the crown of the head of a wearer.

6. A protective mask as in claim 5 wherein said strap means interconnects with and form part of a crown-cushioning pad in crossing over the top of the head of a wearer.

7. A protective mask as in any one of claims 1, 2, 3, 4, 5, or 6 wherein said I goggles incorporate and intimately intermitting frame which frame interengages with said first fastener means.
